(19) United States
(12) Patent Application Publication
(10) Pub. No.: US 2009/0137484 A1
(43) Pub. Date: May 28, 2009

(54) Neuronal Network-Interacting Peptide

(75) Inventors: Jianxing Song, Singapore (SG); Minfen Li, Singapore (SG); Jingxian Liu, Singapore (SG); Xiaoyuan Ran, Singapore (SG)

Correspondence Address: Donald R Studebaker;NIXON PEABODY, Suite 900, 401 9th Street NW, Washington, DC, 20004-2128, US

(73) Assignee: NATIONAL UNIVERSITY OF SINGAPORE, Singapore (SG)

(21) Appl. No.: 11/791,958

(22) PCT Filed: Dec. 1, 2005
(86) PCT No.: PCT/SG05/00411

§ 371 Date: May 31, 2007

Related U.S. Application Data
(60) Provisional application No. 60/632,045, filed on Dec. 1, 2004.

(51) Int. Cl. A61K 38/08; C07K 14/00; C07K 7/00; C12N 15/11; C12N 5/06; C12Q 1/02; G01N 33/00; C12N 15/00; C12N 1/21; C12P 21/04

(52) U.S. Cl. 514/15; 530/350; 530/327; 530/328; 536/23.5; 435/320.1; 435/252.33; 435/69.1; 435/375; 436/86; 435/29

(57) ABSTRACT

The invention provides a method for treating a neuronal-associated condition comprising administering a composition comprising a therapeutically effective amount of a polypeptide or a biologically active fragment thereof, the polypeptide or the fragment thereof capable of modulating the interaction between Nogo-ephrinB and/or Nogo-Grb4 third SH3 domain. There are also provided isolated polypeptide or fragment thereof. The invention further provides a method of screening a compound for effectiveness as an agonist or as an antagonist of the polypeptide or fragment thereof according to the invention.

### FIELD OF THE INVENTION

The present invention is in the field of molecular biology and genetics. More specifically, it relates to the inhibition of nucleic acid and protein sequences of Nogo and Grb4. In particular, the invention relates to an isolated peptide or fragment thereof for use in the treatment of neuronal-related conditions. For example, for treatment of neuronal development and regeneration, angiogenesis.

### BACKGROUND OF THE INVENTION

Two key signalling networks mediated respectively by Nogo and Eph receptor ephrinB play essential roles in neuronal development and regeneration, as well as angiogenesis.

Nogo has been identified as a component of the central nervous system myelin that prevents axonal regeneration in adult vertebrates. Binding interaction between the Nogo-66 (66-residue domain of Nogo) receptor and Nogo molecules as well as other glycophosphatidylinositol-linked proteins from axonal surfaces render neurons insensitive to Nogo-66, facilitating potential recovery from CNS damage (Fournier, A. E. et al. (2001) Nature 409:341-346). The human Nogo has several splicing variants which have received intense attention recently because they are engaged in a variety of critical cellular processes. For example, Nogo-A, the largest splicing variant consisting of 1192 residues, is involved in inhibiting neurite growth (Oertle T, Schwab M E. (2003) Trends Cell Biol. 13, 187-94; Lee D H, et al., (2003) Nat Rev Drug Discov. 2, 872-8; Li M, et al., (2004) Eur J. Biochem. 271, 3512-22), regulating autoimmunemediated demyelination (Karnezis T. et al., (2004) Nat. Neurosci. 7, 736-44) and interacting with BACE1 (He W, et al., (2004) Nat. Med. 10, 959-65) while another variant Nogo-B, which shares the first 200 residues with Nogo-A was identified as a regulator of vascular remodelling (Acevedo L, et al., (2004) Nat. Med. 10, 382-8 and might also be a novel apoptosis-inducer (Li Q, et al., (2001) Oncogene. 20, 3929-36).

On the other hand, Eph-ephrinB mediated signalling network functions at the interface between pattern development and morphogenesis. Recently this signalling network was demonstrated to regulate cytoskeletal remodelling via phosphorylation-dependent binding with the SH2 domain of the Grb4 protein (Cowan C A, Henkemeyer M. (2001) Nature. 413, 174-9; Song J. (2003) J Biol Chem. 278, 24714-20). Grb4, an adaptor protein containing one SH2 and three SH3 domains, appears to serve as a pivotal point to integrate a variety of extracellular signals from cell surface Eph receptor tyrosine kinases and ephrins to downstream effectors mainly controlling cytoskeletal dynamics in a ‘pY-->SH2/SH3-->effector’ connection manner (Cowan C A, Henkemeyer M. (2001) Nature. 413, 174-9; Cowan C A, Henkemeyer M. (2002) Trends Cell Biol. 12, 339-46; Tu Y, Li F, Wu C. (1998) Mol Biol Cell. 9, 3367-82; Li W, et al., (2001) Oncogene. 20, 6403-17;).

Eph receptor-ephrinB mediating signalling pathway has been considered to be implicated in inhibiting CNS neuronal regeneration, but the underlying molecular mechanism still remains completely unclear despite extensive efforts (Fournier A E, Strittmatter S M. (2001) Curr Opin Neurobiol. 11, 89-94; Sandvig A, Berry M, Barrett L B, Butt A, Logan A. (2004) Glia. 46, 225-51).

To date, no evidence has been found which might indicate the presence of a cross-talk or interaction between signalling networks mediated by Nogo and ephrinB or Grb4. There is therefore still a need for further investigation, in this field of science, for a better understanding of the mechanisms of activity of Nogo and/or ephrinB (or Grb4).

### SUMMARY OF THE INVENTION

The present invention has addressed the problems above, and surprisingly the inventors found a binding interaction between the Nogo and Grb4 SH3 domain. This finding provides a mechanism for Nogo-ephrinB communication and for application of intervening molecules of significant therapeutic interest.

According to a first aspect, the present invention provides an isolated polypeptide, wherein the polypeptide is selected from the group consisting of:
(a) a fragment of Nogo-A protein, the fragment being capable of inhibiting the interaction between Nogo-ephrinB and/or Nogo-Grb4 third SH3 domain;
(b) a polypeptide comprising amino acids 1-200 of human Nogo-A or a biologically active fragment thereof capable of inhibiting the interaction between Nogo-ephrinB and/or Nogo-Grb4 third SH3 domain;
(c) a fragment of Nogo-A protein, the fragment comprising the amino acids 1-200 of SEQ ID NO:30;
(d) a polypeptide comprising amino acids 32-200 of human Nogo-A or a biologically active fragment thereof capable of inhibiting the interaction between Nogo-ephrinB and/or Nogo-Grb4 third SH3 domain;
(e) a fragment of Nogo-A protein, the fragment comprising amino acids 32-200 of SEQ ID NO:30;
(f) a polypeptide fragment comprising the amino acid sequence X³X⁵[P]_{z}X³X¹, the fragment being capable of inhibiting the interaction between Nogo-ephrinB and/or Nogo-Grb4 third SH3 domain;
(g) a polypeptide fragment comprising the amino acid sequence of SEQ ID NO:3, the fragment capable of inhibiting the interaction between Nogo-ephrinB and/or Nogo-Grb4 third SH3 domain;
(h) a polypeptide having the amino acid sequence of [Σ]ₙ X³X⁵[P]_{z}X³X¹[U]ₘ;
(i) a polypeptide fragment comprising the amino acid sequence X¹X²PX³X³PX⁴X⁵X⁵X⁶X¹, the fragment capable of inhibiting the interaction between Nogo-ephrinB and/or Nogo-Grb4 third SH3 domain;
(j) a polypeptide fragment comprising the amino acid sequence SEQ ID NO:1, the fragment capable of inhibiting the interaction between Nogo-ephrinB and/or Nogo-Grb4 third SH3 domain; and
(k) a polypeptide having the amino acid sequence of [Σ]ₙX¹X²PX³X³PX⁴X⁵X⁵X⁶X¹[U]ₘ or a biologically active fragment thereof,
   wherein each of X¹ independently is any hydrophilic amino acid; X² is any hydrophilic amino acid; each of X³ independently is a hydrophobic amino acid or Y; X⁴ is any basic amino acid; each of X⁵ independently is any basic amino acid; X⁶ is any hydrophilic amino acid; [Σ] is any amino acid, and n is an integer from 0 to 20; [U] is any amino acid, and m is an integer from 0 to 20; and z is an integer from 1 to 6.

In particular, there is provided an isolated polypeptide having the amino acid sequence of [Σ]ₙX¹X²PX³X³PX⁴X⁵X⁵X⁶X¹[U]ₘ, X³X⁵[P]_{z}X³X¹[U]ₘ or a biologically active fragment thereof, wherein each of X¹ independently may be any hydrophilic amino acid, preferably S; X² may be any hydrophilic amino acid, preferably T; each of X³ independently may be a hydrophobic amino acid or Y, preferably A; X⁴ may be any basic amino acid, preferably, K; each of X⁵ independently may be any basic amino acid, preferably R; X⁶ may be any hydrophilic amino acid, preferably G; [Σ] may be any amino acid and n is an integer from 0 to 20; [U] may be any amino acid and m is n integer from 0 to 20. For example, m and/or n may be an integer from 0 to 20, from 1 to 19, from 2 to 18, from 3 to 17, from 4 to 16, from 5 to 15, from 6 to 14, from 7 to 13, from 8 to 12, from 9 to 11, or from 10 to 11; and z is an integer from 1 to 6. More in particular, the amino sequence of [Σ]ₙX¹X²PX³X³PX⁴X⁵X⁵X⁶X¹[U]ₘ is the amino acid sequence of SEQ ID NO:1. This polypeptide and/or biologically fragment thereof is agent useful in the modulation of Nogo-ephrinB network communication. Further, this agent is useful in therapeutic applications of several conditions like, neuronal-related conditions. In particular, there is also provided the polypeptide X³X⁵PPPPPPX³X¹.

As mentioned above, the polypeptide of the invention encompasses the amino acid sequence of SEQ ID NO:1, or the amino acid sequence of SEQ ID NO:3 or a fragment thereof with any conservative amino acid amino acid substitution, modification and/or variation which does not affect the modulation activity of Nogo-ephrinB network communication. The polypeptide of the invention may have a binding affinity to Grb4.

According to another aspect, the invention provides a polynucleotide comprising an oligonucleotide encoding the any polypeptide according to the invention. In particular, a polynucleotide comprising an oligonucleotide encoding any polypeptide (a) to (k) as above described. In particular, there is provided polynucleotide comprising an oligonucleotide encoding a polypeptide having the amino acid sequence of [Σ]ₙX¹X²PX³X³PX⁴X⁵X⁵X⁶X¹[U]ₘ, the amino acid sequence X³X⁵[P]_{z}X³X¹, or a biologically active fragment thereof. More in particular, the invention provides a polynucleotide comprising an oligonucleotide encoding the peptide of SEQ ID NO:1, the peptide SEQ ID NO:3, or a biologically active fragment thereof.

The invention also provides a recombinant polynucleotide comprising a promoter sequence operably linked to the polynucleotide of the invention.

The invention also provides a vector comprising the polynucleotide of the invention.

The invention also provides a host cell transformed with the polynucleotide according to any embodiment of the invention and/or the vector of the invention.

The invention also provides a method of producing a polypeptide or the fragment thereof according to the invention, the method comprising: a) culturing a cell under conditions suitable for expression of the polypeptide or fragment thereof, wherein said cell is transformed with a vector comprising a polynucleotide encoding the polypeptide or fragment thereof, and b) recovering the polypeptide or fragment thereof so expressed.

There is also provided a composition comprising the polypeptide or fragment thereof of any embodiment of the invention. The composition may be a pharmaceutical composition. The composition may further comprise a pharmaceutically acceptable diluent, carrier, excipient or a combination thereof.

According to another aspect, the invention provides a method of modulating the Nogo-ephrinB network interaction and/or Nogo-Grb4 third SH3 domain interaction comprising adding a compound to a cell in vitro or in vivo; the compound being capable of modulating the Nogo-ephrinB network interaction and/or Nogo-Grb4 third SH3 domain interaction. In particular, the addition of the compound inhibits the interaction between Nogo-ephrinB and/or Nogo-Grb4 third SH3 domain. The compound is, in particular, a polypeptide or a biologically active fragment thereof. More in particular, a polypeptide or fragment thereof according to any embodiment of the invention.

According to another aspect, the invention provides a method for treating neuronal- and angiogenesis-associated conditions comprising administering to a subject in need a composition comprising a therapeutically effective amount of a compound capable of modulating the interaction between Nogo-ephrinB and/or Nogo-Grb4 third SH3 domain. In particular, the compound is capable of inhibiting the interaction between Nogo-ephrinB and/or Nogo-Grb4 third SH3 domain. More in particular, the compound is a polypeptide or a biologically active fragment thereof, preferably the polypeptide or fragment thereof according to any embodiment of the present invention.

The neuronal- and angiogenesis-associated condition may be at least one condition selected from the group consisting of: control of neuronal development, neuronal regeneration, angiogenesis, inhibition of neurite growth, regulating autoimmune-mediated demyelination, regulator of vascular remodelling, apoptosis-inducer.

The subject is preferably a mammal, in particular a human.

According to another aspect, the invention provides a method of screening a compound for effectiveness as an agonist or as an antagonist of a polypeptide or fragment thereof according to the invention, the method comprising: a) exposing a sample comprising a polypeptide or fragment thereof, according to the invention, to a compound; and b) detecting agonist or antagonist activity in the sample.

According to another aspect, the invention provides a method of screening a compound for effectiveness as a modulator of Nogo-ephrinB and/or Nogo-Grb4 third SH3 domain interaction, the method comprising a) adding a test compound to a cell in vitro or in vivo; and b) detecting the modulating activity of the test compound. The method may further comprise the steps of: c) adding the polypeptide or a fragment thereof according to the invention to a cell in vitro or in vivo; d) detecting the modulating activity of the polypeptide or fragment thereof; and e) comparing the modulating activity of the test compound with the modulating activity of the polypeptide or a fragment thereof.

According to another aspect, the invention relates to the polypeptide or a biologically active fragment thereof for use in medicine.

According to another aspect, the invention relates to the use of the polypeptide or a biologically active fragment thereof for the preparation of a medicament for the treatment of neuronal-associated conditions.

### BRIEF DESCRIPTION OF THE FIGURES AND THE SEQUENCE LISTINGS

FIG. 1(a, b, c, and d). Identification of the binding interface between the 11-residue Nogo-A (171-181) and the third Grb4 SH3 domain. (a) The NMR structure of the third Grb4 SH3 domain showing the conserved SH3 fold with secondary structure elements and critical regions for binding labelled. (b) Superimposition of two-dimensional 1H-15N NMR HSQC spectra of the third Grb4 SH3 domain in the absence (also indicated in blue in the figure in colour) and in the presence (also indicated in red in the figure in colour) of 2-fold excess of the Nogo-A (171-181) peptide. The resonance assignments of the HSQC peaks with significant perturbations were labelled. (c)-(d) The NMR structures of the third Grb4 SH3 domain in two different orientations with the residues whose HSQC peaks underwent significant shifts coloured in red.

FIG. 2. Two-dimensional ¹H-¹⁵N NMR spectra of the ¹⁵N-isotope labeled Grb4 [115-257] at pH 6.8 and 20° C. in the absence (blue) and in the presence (red) of the Nogo-A[1-200] protein.

SEQ ID NO: 1:
STPAAPKRRGS (corresponding to the fragment 171-181
of Nogo A).
SEQ ID NO: 2:
APPVAPERQPS (corresponding to the fragment 97-107
of Nogo A).
SEQ ID NO: 3:
ARPPPPPPAS (corresponding to the fragment 141-150
of Nogo A).
SEQ ID NO: 4:
GTG ATT GTG ATT GCG AAA TGG GAT TAT ACC GCG
CAG CAG GAT CAG GAA CTG GAT;
SEQ ID NO: 5:
ATC ATC CAG CAG CCA CAG ACG TTC GTT CAC TTT
TTT AAT ATC CAG TTC CTG ATC;
SEQ ID NO: 6:
TGG CTG CTG GAT GAT AGC AAA ACC TGG TGG CGT
GTG CGT AAC GCG GCG AAC CGT;
SEQ ID NO: 7:
GTT TTT ACG TTC CAC ATA GTT GCT CGG CAC ATA
GCC GGT ACG GTT CGC CGC GTT;
SEQ ID NO: 8:
CAG AAC GCG GGG GGA TCC GTG ATT GTG ATT GCG
AAA;
SEQ ID NO: 9:
CAG AAC CCG CTC GAG TCA GTT TTT ACG TTC CAC
ATA;
SEQ ID NO: 10:
ATT CCG GCG TTT GTG AAA TTT GCG TAT GTG GCG
GAA CGT GAA GAT GAA CTG AGC;
SEQ ID NO: 11:
GCA TTT TTC CAT CAC GGT CAC ACG GCT GCC TTT
CAC CAG GCT CAG TTC ATC TTC;
SEQ ID NO: 12:
GTG ATG GAA AAA TGC AGC GAT GGC TGG TGG CGT
GGC AGC TAT AAC GGC CAG ATT;
SEQ ID NO: 13:
ATC CAC TTC TTC CAG CAC ATA GTT GCT CGG AAA
CCA GCC AAT CTG GCG GTT ATA;
SEQ ID NO: 14:
CAG AAC GCG GGG GGA TCC ATT CCG GCG TTT GTG
AAA;
SEQ ID NO: 15:
CAG AAC CCG CTC GAG TCA ATC CAC TTC TTC CAG
CAC;
SEQ ID NO: 16:
CAT GTG GTG CAG ACC CTG TAT CCG TTT AGC AGC
GTG ACC GAA GAA GAA CTG AAC;
SEQ ID NO: 17:
CGG TTT TTC AAT CAC TTC CAT GGT TTC GCC TTT
TTC AAA GTT CAG TTC TTC TTC;
SEQ ID NO: 18:
GTG ATT GAA AAA CCG GAA AAC GAT CCG GAA TGG
TGG AAA TGC AAA AAC GCG CGT;
SEQ ID NO: 19:
GCT CAG CAC CAC CAC ATA GTT TTT CGG CAC CAG
GCC CAC CTG GCC ACG CGC GTT TTT GCA;
SEQ ID NO: 20:
CAG AAC GCG GGG GGA TCC CAT GTG GTG CAG
ACC CTG;
SEQ ID NO: 21:
CAG AAC CCG CTC GAG TCA GCT CAG CAC CAC CAC
ATA;
SEQ ID NO: 22:
CGC GCC TTT ACG CAG GCT CAG AAA GCT CGG GCT
TTC CGC CGC CGC TTC ATC CAC TTC TTC CAG;
SEQ ID NO: 23:
CTG CGT AAA GGC GCG AGC CTG AGC AAC GGC
CAG GGC AGC CGT GTG CTG CAT GTG GTG CAG ACC;
SEQ ID NO: 24:
CGC GCG CGC GGA TCC ATG GAA GAC CTG GAC
CAG;
SEQ ID NO 25:
CGC GCG CGC CTC GAG CTA TAT CAC AGG CTC AGA
TGC;
SEQ ID NO: 26:
CGC GCG CGC GGA TCC GAG GAC GAG GAG GAA;
SEQ ID NO: 27:
CGC GCG CGC CTC GAG CTA GAA GTC ATT TCC GAA
GTC;
SEQ ID NO: 28:
CGC GCG CGC GGA TCC TTC GTG CCG CCG GCG
CCC;
SEQ ID NO: 29:
CGC GCG CGC CTC GAG CTA TAT CAC AGG CTC AGA
TGC.
SEQ ID NO: 30:
sequence of residues 1 to 1192 of Nogo-A human
protein obtainable from NCBI web site
(http://ncbi.nlm.nih.gov) identified with the
code CAB99248.

### DETAILED DESCRIPTION OF THE INVENTION

Before the present polypeptides, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and equivalents thereof known to those skilled in the art, and so forth.

Unless defined otherwise, all technical and scientific terms used herein have the same meaning as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### DEFINITIONS AND NOMENCLATURE

The terminology used herein is for the purpose of describing particular embodiments only and is not intended to be limiting in nature.

The amino acid abbreviations used herein are conventional one letter codes for the amino acids and are expressed as follows: A or Ala, alanine; C or Cys, cysteine; D or Asp, aspartic acid; E or Glu, glutamic acid; F or Phe, phenylalanine; G or Gly, glycine; H or H is, histidine; I or Ile, isoleucine; K or Lys, lysine; L or Leu, leucine; M or Met, methionine; N or Asn, asparagine; P or Pro, proline; Q or Gln, glutamine; R or Arg, arginine; S or Ser, serine; T or Thr, threonine; V or Val, valine; W or Trp, tryptophan; Y or Tyr, tyrosine; Z, glutamine or glutamic acid.

A “conservative amino acid substitution” is one in which the amino acid residue is replaced with an amino acid residue having a similar side chain. Families of amino acid residues having similar side chains have been defined in the art. These families include hydrophobic, hydrophilic, basic and acid amino acids as described below. Thus, a predicted nonessential amino acid residue in the peptide of the invention is preferably replaced with another amino acid residue from the same category. For example, a hydrophobic amino acid is replaced by another hydrophobic amino acid, etc. Alternatively, in another embodiment, mutations can be introduced randomly along all or part of the coding sequence of the nucleic acid encoding the peptide of the invention, such as by saturation mutagenesis, and the resultant mutants can be screened for anticoagulant activity to identify mutants that retain that activity.

“Hydrophobic” amino acids are: A, V, L, I, P, W, F and M; “hydrophilic” amino acids are: G, S, T, Y, C, N and Q; however Y has a hydrophobic aromatic group and may be considered to be acting as a hydrophobic amino acid depending on the circumstances and uses; “basic” amino acids are: K, R and H; and “acidic” amino acids are: D and E.

“Polypeptide” as used herein refers to and encompasses any amino acid molecule, a peptide or polypeptide. The “polypeptide” can be obtained as a gene product, a purified and/or isolated product, an expression product, from fragmentation of protein(s) or a synthetic peptide. An “isolated polypeptide” encompasses naturally occurring, a gene expression product and a synthetic peptide. The “polypeptide” as herein used also encompasses any fragment thereof which maintain the activity of the “polypeptide”. For the purpose of the present description, the fragment will be indicated as “a biologically active fragment thereof” or simply “a fragment thereof”.

The phrase “nucleic acid” or “nucleic acid molecule” as used herein refers to a naturally occurring or synthetic oligonucleotide or polynucleotide, whether DNA or RNA or DNA-RNA hybrid, single-stranded or double-stranded, sense or antisense, which is capable of hybridisation to a complementary nucleic acid by Watson-Crick base-pairing. Nucleic acids of the invention can also include nucleotide analogues (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

As used herein, the term “agonist” refers to a molecule which, when bound to the polypeptide or fragment thereof according to the invention, causes a change in the polypeptide or fragment thereof which stimulates or mimics the activity of the polypeptide or fragment thereof. Agonists may include proteins, nucleic acids, carbohydrates, or any other molecule that binds to the polypeptide. More in particular, an agonist according to the present invention stimulates, in the presence of the polypeptide or fragment of the invention, the inhibiting interaction between Nogo-ephrinB and/or Nogo-Grb4 third SH3 domain.

The term “antagonist”, as used herein, refers to a molecule which, when bound to the polypeptide or fragment of the invention, blocks, inhibits, reduces or attenuates the biological or immunological activity of the polypeptide or fragment of the invention. Antagonists and inhibitors may include proteins, nucleic acids, carbohydrates, or any other molecules which bind to the polypeptide.

“Amplification” relates to the production of additional copies of a nucleic acid. Amplification may be carried out using the polymerase chain reaction (PCR) technologies or any other nucleic acid amplification technologies well known in the art.

### Compounds, Compositions, Uses and Methods of the Invention

The authors of the present invention have identified Grb4 as a binding partner of Nogo-A N-terminal domain, thus establishing an alternative pathway for Nogo signalling other than Nogo-66-NogoR interaction. The present inventors have identified and validated that the specific interaction between Nogo and Grb4 provides a molecular mechanism and thus will facilitate further in vitro and/or in vivo investigations of the cross-communication between Nogo and ephrinB mediated signalling networks. In particular, the compound(s) disclosed herein have a therapeutic application of conditions which are modulated by Nogo and ephrinB signalling networks, for instance, in neuronal-associated conditions and may also be used to develop further therapeutic agents to intervene in mammal, including human, diseases.

The sequence of residues 1 to 1192 of Nogo-A human protein obtainable from NCBI web site (http://ncbi.nlm.nih.gov) identified with the code CAB99248 is reported in SEQ ID NO:30.

According to one aspect, the invention related to fragment(s) of Nogo-A polypeptide. In particular, fragment(s) of the polypeptide having the sequence SEQ ID NO:30. In particular, the invention relates to polypeptide(s) or fragment(s) thereof being capable of inhibiting the interaction between Nogo-ephrinB and/or Nogo-Grb4 third SH3 domain

According to one aspect, the present invention provides an isolated polypeptide, wherein the polypeptide is selected from the group consisting of:
(a) a fragment of Nogo-A protein, the fragment being capable of inhibiting the interaction between Nogo-ephrinB and/or Nogo-Grb4 third SH3 domain;
(b) a polypeptide comprising amino acids 1-200 of human Nogo-A or a biologically active fragment thereof capable of inhibiting the interaction between Nogo-ephrinB and/or Nogo-Grb4 third SH3 domain;
(c) a fragment of Nogo-A protein, the fragment comprising the amino acids 1-200 of SEQ ID NO:30;
(d) a polypeptide comprising amino acids 32-200 of human Nogo-A or a biologically active fragment thereof capable of inhibiting the interaction between Nogo-ephrinB and/or Nogo-Grb4 third SH3 domain;
(e) a fragment of Nogo-A protein, the fragment comprising amino acids 32-200 of SEQ ID NO:30;
(f) a polypeptide fragment comprising the amino acid sequence X³X⁵[P]_{z}X³X¹, the fragment being capable of inhibiting the interaction between Nogo-ephrinB and/or Nogo-Grb4 third SH3 domain;
(g) a polypeptide fragment comprising the amino acid sequence of SEQ ID NO:3, the fragment capable of inhibiting the interaction between Nogo-ephrinB and/or Nogo-Grb4 third SH3 domain;
(h) a polypeptide having the amino acid sequence of [Σ]ₙX³X⁵[P]_{z}X³X¹[U]ₘ;
(i) a polypeptide fragment comprising the amino acid sequence X¹X²PX³X³PX⁴X⁵X⁵X⁶X¹, the fragment capable of inhibiting the interaction between Nogo-ephrinB and/or Nogo-Grb4 third SH3 domain;
(j) a polypeptide fragment comprising the amino acid sequence SEQ ID NO:1, the fragment capable of inhibiting the interaction between Nogo-ephrinB and/or Nogo-Grb4 third SH3 domain; and
(k) a polypeptide having the amino acid sequence of [Σ]ₙX¹X²PX³X³PX⁴X⁵X⁵X⁶X¹ [U]ₘ or a biologically active fragment thereof,
   wherein each of X¹ independently is any hydrophilic amino acid; X² is any hydrophilic amino acid; each of X³ independently is a hydrophobic amino acid or Y; X⁴ is any basic amino acid; each of X⁵ independently is any basic amino acid; X⁶ is any hydrophilic amino acid; [Σ] is any amino acid, and n is an integer from 0 to 20; [U] is any amino acid, and m is an integer from 0 to 20; and z is an integer from 1 to 6.

Accordingly, a fragment of Nogo-A protein, for example any fragment shown in Table 1 refers to the sequence of SEQ ID NO:30. For example, a fragment of Nogo-A protein which comprises or has the amino acid sequence of 1-200, 32-200, 141-150 or 171-181 amino acids of Nogo-A refers to a fragment of the protein of SEQ ID NO:30.

In particular, the present invention provides this agent is a polypeptide having the amino acid sequence of [Σ]ₙX¹X²PX³X³PX⁴X⁵X⁵X⁶X¹[U]ₘ, X³X⁵[P]_{z}X³X¹[U]ₘ or a biologically active fragment thereof, wherein each of X¹ independently may be any hydrophilic amino acid, preferably S; X² may be any hydrophilic amino acid, preferably T; each of X³ independently may be a hydrophobic amino acid or Y, preferably A; X⁴ may be any basic amino acid, preferably, K; each of X⁵ independently may be any basic amino acid, preferably R; X⁶ may be any hydrophilic amino acid, preferably G; [Σ] may be any amino acid, and n is an integer from 0 to 20; [U] may be any amino acid and m is an integer from 0 to 20, and z is an integer from 1 to 6. More in particular, n may be an integer from 0 to 20, from 1 to 19, from 2 to 18, from 3 to 17, from 4 to 16, from 5 to 15, from 6 to 14, from 7 to 13, from 8 to 12, from 9 to 11 or from 10 to 11. Further, m may be 0 to 20, from 1 to 19, from 2 to 18, from 3 to 17, from 4 to 16, from 5 to 15, from 6 to 14, from 7 to 13, from 8 to 12, from 9 to 11 or from 10 to 11. Further, z may be an integer corresponding to the value 1, 2, 3, 4, 5 or 6. More in particular, the present invention provides an isolated polypeptide comprising the amino acid sequence STPAAPKRRGS (SEQ ID NO:1), ARPPPPPPAS (SEQ ID NO:3) or a biologically active fragment thereof. This polypeptide may be useful in the modulation of Nogo-ephrinB network communication. Further, this polypeptide may be useful in therapeutic applications of several conditions as described herein. The polypeptide of the invention may be a polypeptide having a binding affinity to Grb4.

The polypeptide of the invention encompasses an amino acid sequence based on the amino acid sequence of SEQ ID NO:1, SEQ ID NO:3 or a biologically active fragment thereof including any conservative amino acid amino acid substitution, modification and/or variation which does not affect its modulation activity of Nogo-ephrinB network communication. In particular, the invention also encompasses an amino acid sequence of [Σ]ₙX¹X²PX³X³PX⁴X⁵X⁵X⁶X¹[U]ₘ, or [Σ]ₙX³X⁵[P]_{z}X³X¹[U]ₘ, wherein one or more amino acid is deleted or added from the sequence. In particular, there is provided a polypeptide having sequence SEQ ID NO:1, or SEQ ID NO:3, wherein one or more amino acid is deleted or added to the sequence of SEQ ID NO:1 or SEQ ID NO:3. Accordingly, substitutions, deletion and/or modifications of the amino acids of the polypeptide comprising the amino acid sequence of SEQ ID NO:1, SEQ ID NO:3 or a fragment thereof may be carried out according to standard technologies. For example, A and P, which are hydrophobic may be substituted with any other hydrophobic amino acid. S, T, G, which are hydrophilic may be substituted with any other hydrophilic amino acids. K and R, which are basic amino acids may be substituted with any other basic amino acid. Substituting amino acids with other amino acids of similar properties is acceptable, as the overall properties of the peptide are not altered.

Polypeptide Production—The polypeptide of the invention may be produced by any method known in the art. One method of producing the disclosed peptides is to link two or more amino acid residues together by protein chemistry techniques. For example, peptides are chemically synthesised using currently available laboratory equipment using either Fmoc (9-fluorenylmethyloxycar-bonyl) or Boc (tert-butyloxycarbonoyl) chemistry (Applied Biosystems, Inc., Foster City, Calif.). A peptide can be synthesised and not cleaved from its synthesis resin, whereas the other fragment of a peptide or protein can be synthesized and subsequently cleaved from the resin, thereby exposing a terminal group, which is functionally blocked on the other fragment. By peptide condensation reactions, these two fragments can be covalently joined via a peptide bond at their carboxyl and amino termini, respectively, (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY). Alternatively, the peptide is independently synthesised in vivo. Once isolated, these independent peptides may be linked to form a peptide or fragment thereof via similar peptide condensation reactions. The polypeptide according to the invention may also be prepared via standard DNA recombinant technologies known in the art, for example, those described in Sambrook and Russel, Molecular Cloning, A laboratory manual, Cold Spring Harbour Laboratory Press, 2001.

According to another aspect, the invention provides a polynucleotide having an oligonucleotide encoding the peptide of amino acid sequence [Σ]ₙX¹X²PX³X³PX⁴X⁵X⁵X⁶X¹[U]ₘ, [Σ]ₙX³X⁵[P]_{z}X³X¹[U]ₘ; or a biologically active fragment thereof. More in particular, the invention provides a polynucleotide comprising an oligonucleotide encoding the peptide of amino acid sequence SEQ ID NO:1, SEQ ID NO:3 or a biologically active fragment thereof. The polynucleotide of the invention may be prepared according to standard techniques in the art. The invention also provides a recombinant polynucleotide comprising a promoter sequence operably linked to the polynucleotide of the invention. Further, the invention also provides a vector comprising the polynucleotide of the invention, as well as a host cell transformed with the polynucleotide, recombinant polynucleotide according to any embodiment of the invention and/or the vector of the invention.

Nucleic Acid and Vectors—The invention is also directed to an isolated nucleic acid encoding any one or more of the peptides disclosed herein. In one embodiment, the nucleic acid comprises DNA, RNA and/or cDNA. It would be routine for one with ordinary skill in the art to make a nucleic acid that encodes the peptides disclosed herein since codons for each of the amino acids that make up the peptides are known. As non-limiting examples, the nucleic acids of the invention can be produced by recombinant, in vitro methods, or by chemical synthetic means using machines and standard chemistry which would be known to one with skill in the art, or by in vivo cellular synthesis. Methods of synthesising nucleic acids would be well known to one with skill in the art. For example, U.S. Pat. No. 6,472,184 entitled “Method for producing nucleic acid polymers” and U.S. Pat. No. 6,444,111 entitled “Electrochemical solid phase synthesis of polymers” describe such synthesis and synthetic methods. These references are hereby incorporated by reference in their entireties.

Additionally, the invention provides a vector comprising the nucleic acid encoding any one or more of the peptides described herein. In certain embodiments, the invention provides a vector comprising a nucleic acid encoding at least one of the peptides of the present invention. The vector can be a viral vector, a plasmid vector, a cosmid vector, an adenoviral vector, a phage vector, a retroviral vector, an adeno-associated viral (MV) vector, or any other vector capable of including a nucleic acid encoding a peptide of the invention. The vector can be an expression vector that is intended and capable of integrating into a cell genome. Among vectors preferred for use in bacteria include pQE70, pQE6 and pQE-9, available from Qiagen; pBS vectors, Phagescript vectors, Bluescript vectors, pNH8A, pNH16a, pNH18A and pNH46A, available from Stratagene; pcDNA3 available from Invitrogen; and pGEX, pTrxfus, pTrc99a, pET-5, pET-9, pKK223-3, pKK233-3. pDR540 and pRIT5 available from Pharmacia. Among preferred eukaryotic vectors are pWLNEO, pSV2CAT, pOG44, pXT1, pBK and pSG available from Stratagene; and pSVK3, pBPV, pMSG and pSVL available from Pharmacia. Other useful virus vectors include retroviruses such as Moloney murine leukemia virus (MoMuLV); papovaviruses such as JC, SV40, polyoma, adenoviruses; Epstein-Barr Virus (EBV); papilloma viruses, e.g. bovine papilloma virus type I (BPV); vaccinia and poliovirus and other human and animal viruses. Other suitable vectors will be readily apparent to the skilled artisan. Useful vectors and their construction are disclosed in Sambrook and Russel, (2001) Molecular Cloning: A Laboratory Manual, Cold Spring Harbor Laboratory Press, US.

Host cell—The invention also provides for a host cell containing the nucleic acid, polypeptide, peptide, and/or the vector of the invention. Such a host cell is a eukaryotic cell or a prokaryotic cell. In the case of eukaryotic cells, retrovirus or adenovirus based vectors can be used to put the nucleic acid of the invention into the host cell. Methods known to one with skill in the art for inserting the nucleic acids or polypeptides in host cells are encompassed within this invention. The following are non-limiting examples of such methods: naked DNA transfection, lipofectin-mediated transfer, transformation, micro-injection of nucleic acid into a cell, or calcium-phosphate precipitation transfection methods. Host cells can be obtained from commercial sources such as the American Type Culture Collection (ATCC). Host cells can be grown in liquid media culture or on tissue culture plates. The growth conditions will be dependent upon the specific host cells used and such conditions would be known to one with skill in the art. Transfection and growth of host cells is described in Sambrook and Russel, id.

The invention provides for a recombinant cell expressing a nucleic acid encoding the polypeptide of the claimed invention. The invention also provides for a recombinant cell producing the polypeptide of the invention.

The method for producing any polypeptide according to the invention also encompasses the method known as “gene activation”, for example as the method developed by Transkaryotic Therapies Inc. (TKT) (see for example the method for the preparation of the TKT's erythropoietin, generally known with the brand name Dynepo), comprising introducing into a cell into an exogenous DNA molecule to stimulate the production of an endogenous polypeptide.

Accordingly, the invention provides a method of producing a polypeptide or the fragment thereof according to the invention, the method comprising: a) culturing a cell under conditions suitable for expression of the polypeptide or fragment thereof, wherein said cell is transformed with a vector comprising a polynucleotide encoding the polypeptide or fragment thereof, —and b) recovering the polypeptide or fragment thereof so expressed.

According to another aspect, the invention provides a composition comprising the polypeptide or a fragment thereof according to the invention, the nucleic acid molecule encoding the polypeptide of the invention, or the vector. In particular, the composition is a pharmaceutical composition. The composition may further comprise a pharmaceutically acceptable excipient, diluent, carrier or a combination thereof.

The formulations of the pharmaceutical compositions described herein may be prepared by any method known or hereafter developed in the art. In general, such preparatory methods include the step of bringing the active ingredient into association with a carrier, excipient and/or diluent, and then, if necessary or desirable, shaping or packaging the product into a desired single- or multi-dose unit.

Although the descriptions of pharmaceutical compositions provided herein are principally directed to pharmaceutical compositions that are suitable for administration to humans, the person skilled in the art will understand that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modifications. Subjects to which administration of the pharmaceutical compositions of the invention is contemplated include, but are not limited to, humans and other primates.

According to another aspect, the invention provides a method of modulating the Nogo-ephrinB network interaction and/or Nogo-Grb4 third SH3 domain interaction comprising adding a compound to a cell in vitro or in vivo; the compound being capable of modulating the Nogo-ephrinB network interaction and/or Nogo-Grb4 third SH3 domain interaction. In particular, the addition of the compound inhibits the interaction between Nogo-ephrinB and/or Nogo-Grb4 third SH3 domain. The compound is in particular a polypeptide or a biologically active fragment thereof. More in particular a polypeptide or fragment thereof according to the invention.

According to another aspect, the invention provides a method for treating neuronal- and angiogenesis-associated conditions comprising administering to a subject in need a composition comprising a therapeutically effective amount of a compound capable of modulating the interaction between Nogo-ephrinB and/or Nogo-Grb4 third SH3 domain. In particular, the compound is capable of inhibiting the interaction between Nogo-ephrinB and/or Nogo-Grb4 third SH3 domain. More in particular, the compound is a polypeptide or a biologically active fragment thereof, preferably the polypeptide or fragment thereof according to the invention.

The neuronal- and angiogenesis-associated condition may be at least one condition selected from the group consisting of: control of neuronal development, neuronal regeneration, angiogenesis, inhibition of neurite growth, regulating autoimmune-mediated demyelination, regulator of vascular remodelling, apoptosis-inducer.

The subject is preferably a mammal, in particular a human.

According to another aspect, the invention provides a method of screening a compound for effectiveness as an agonist or as an antagonist of a polypeptide or fragment thereof according to the invention, the method comprising: a) exposing a sample comprising a polypeptide or fragment thereof, according to the invention, to a compound; and b) detecting agonist or antagonist activity in the sample.

According to another aspect, the invention provides a method of screening a compound for effectiveness as a modulator of Nogo-ephrinB and/or Nogo-Grb4 third SH3 domain interaction, the method comprising a) adding a test compound to a cell in vitro or in vivo; and b) detecting the modulating activity of the test compound. The method may further comprise the steps of: c) adding the polypeptide or a fragment thereof according to the invention to a cell in vitro or in vivo; d) detecting the modulating activity of the polypeptide or fragment thereof; and e) comparing the modulating activity of the test compound with the modulating activity of the polypeptide or a fragment thereof.

According to another aspect, the invention relates to the polypeptide or a biologically active fragment thereof for use in medicine.

According to another aspect, the invention relates to the use of the polypeptide or a biologically active fragment thereof for the preparation of a medicament for the treatment of neuronal-associated conditions.

The invention described herein is susceptible to variations, modifications and or additions other than those specifically described and it is to be understood that the invention includes any such variations, modifications and or additions which fall within the scope of the following claims.

Having now generally described the invention, the same will be more readily understood through reference to the following examples which are provided by way of illustration, and are not intended to be limiting of the present invention.

### EXAMPLES

### Example 1

### Preparation of Grb4, Nogo Proteins and Peptides

The DNA fragments encoding the first, second and third isolated SH3 domains corresponding to the human Grb4 residues 5-61; 115-171 and 199-257 respectively were synthesized based on the human Grb4 sequence (Genbank Accession Number: AAH07195) with E. coli preferred codon usage using a PCR-based strategy (Primers for human Grb4 residues 5-61:

(SEQ ID NO: 4)

| | |
|---|---|
| SH1-1: | GTG ATT GTG ATT GCG AAA TGG GAT TAT ACC GCG |
| | CAG CAG GAT CAG GAA CTG GAT; |

(SEQ ID NO: 5)

| | |
|---|---|
| SH1-2: | ATC ATC CAG CAG CCA CAG ACG TTC GTT CAC TTT |
| | TTT AAT ATC CAG TTC CTG ATC; |

(SEQ ID NO: 6)

| | |
|---|---|
| SH1-3: | TGG CTG CTG GAT GAT AGC AAA ACC TGG TGG CGT |
| | GTG CGT AAC GCG GCG AAC CGT; |

(SEQ ID NO: 7)

| | |
|---|---|
| SH1-4: | GTT TTT ACG TTC CAC ATA GTT GCT CGG CAC ATA |
| | GCC GGT ACG GTT CGC CGC GTT |

(SEQ ID NO: 8)

| | |
|---|---|
| SH1-5: | CAG AAC GCG GGG GGA TCC GTG ATT GTG ATT GCG |
| | AAA; |

(SEQ ID NO: 9)

| | |
|---|---|
| SH1-6: | CAG AAC CCG CTC GAG TCA GTT TTT ACG TTC CAC |
| | ATA; |

Primers for Grb4 residues 115-171

(SEQ ID NO: 10)

| | |
|---|---|
| SH2-1: | ATT CCG GCG TTT GTG AAA TTT GCG TAT GTG GCG |
| | GAA CGT GAA GAT GAA CTG AGC; |

(SEQ ID NO: 11)

| | |
|---|---|
| SH2-2: | GCA TTT TTC CAT CAC GGT CAC ACG GCT GCC TTT |
| | CAC CAG GCT CAG TTC ATC TTC; |

(SEQ ID NO: 12)

| | |
|---|---|
| SH2-3: | GTG ATG GAA AAA TGC AGC GAT GGC TGG TGG CGT |
| | GGC AGC TAT AAC GGC CAG ATT; |

(SEQ ID NO: 13)

| | |
|---|---|
| SH2-4: | ATC CAC TTC TTC CAG CAC ATA GTT GCT CGG AAA |
| | CCA GCC AAT CTG GCC GTT ATA; |

(SEQ ID NO: 14)

| | |
|---|---|
| SH2-5: | CAG AAC GCG GGG GGA TCC ATT CCG GCG TTT GTG |
| | AAA; |

(SEQ ID NO: 15)

| | |
|---|---|
| SH2-6: | CAG AAC CCG CTC GAG TCA ATC CAC TTC TTC CAG |
| | CAC; |

and Primers for Grb4 residues 199-257):

(SEQ ID NO: 16)

| | |
|---|---|
| SH3-1: | CAT GTG GTG CAG ACC CTG TAT CCG TTT AGC AGC |
| | GTG ACC GAA GAA GAA CTG AAC; |

(SEQ ID NO: 17)

| | |
|---|---|
| SH3-2: | CGG TTT TTC AAT CAC TTC CAT GGT TTC GCC TTT |
| | TTC AAA GTT CAG TTC TTC TTC; |

(SEQ ID NO: 18)

| | |
|---|---|
| SH3-3: | GTG AH GAA AAA CCG GAA AAC GAT CCG GAA TGG |
| | TGG AAA TGC AAA AAC GCG CGT; |

(SEQ ID NO: 19)

| | |
|---|---|
| SH3-4: | GCT CAG CAC CAC CAC ATA GTT TTT CGG CAC CAG |
| | GCC CAC CTG GCC ACG CGC GTT TTT GCA; |

(SEQ ID NO: 20)

| | |
|---|---|
| SH3-5: | CAG AAC GCG GGG GGA TCC CAT GTG GTG CAG ACC |
| | CTG; |

(SEQ ID NO: 21)

| | |
|---|---|
| SH3-6: | CAG AAC CCG CTC GAG TCA GCT CAG CAC CAC CAC |
| | ATA. |

The obtained SH3 domains were further linked together by PRC reaction to introduce the connection regions in the Grb4 sequence with nucleotide primers CGC GCC TTT ACG CAG GCT CAG AAA GCT CGG GCT TTC CGC CGC CGC TTC ATC CAC TTC TTC CAG (SEQ ID NO:22)

### and CTG CGT AAA GGC GCG AGC CTG AGC AAC GGC CAG GGC AGC CGT GTG CTG CAT GTG GTG CAG ACC (SEQ ID NO:23).

The DNA fragments were inserted into the Pgex-4T-1 (Pharmacia)/Pet-3 (Novagen) vectors with BamHI and XhoI restriction sites and the DNA sequences were confirmed by automated DNA sequencing (ABI). The recombinant proteins were expressed in E. coli BL21 cells (Novagen) with induction by IPTG (Invitrogen) (isopropyl-beta-D-thiogalactopyranoside) as His-tagged proteins which were purified by Ni-NTA affinity columns (QIAGEN) according to the protocol provided by the manufacture followed by HPLC on a reversed phase C8 column (Vydac). The DNA fragments encoding the human Nogo-A (1-200), Nogo-A (32-85) and Nogo-A (32-200) were obtained by PCR with three pairs of primers: 5′-CGC GCG CGC GGA TCC ATG GAA GAC CTG GAC CAG-3′ (SEQ ID NO:24) and 5′-CGC GCG CGC CTC GAG CTA TAT CAC AGG CTC AGA TGC-3′ (SEQ ID NO:25) for Nogo-A (1-200); 5′-CGC GCG CGC GGA TCC GAG GAC GAG GAG GAA-3′ (SEQ ID NO:26) and 5′-CGC GCG CGC CTC GAG CTA GAA GTC ATT TCC GAA GTC-3′ (SEQ ID NO:27) for Nogo-A (32-85) and 5′-CGC GCG CGC GGA TCC TTC GTG CCG CCG GCG CCC-3′ (SEQ ID NO:28) and 5′-CGC GCG CGC CTC GAG CTA TAT CAC AGG CTC AGA TGC-3 (SEQ ID NO:29) for Nogo-A (32-200) on a Nogo-A cDNA, The PCR fragments were subsequently cloned into the expression vector pET32a (Novagen) with BamHI/XhoI restriction sites. Procedures similar to those used for the preparation of the Grb4 proteins were utilised to generate Nogo proteins except that a reversed phase C4 column (Vydac) was used for purification. For isotope-labelling, recombinant proteins were prepared by growing the BL21 cells in the M9 medium prepared by adding (¹⁵NH₄)₂SO₄ (Isotope-Cambridge) for ¹⁵N labelling and (¹⁵NH₄)₂SO₄, ¹³C-glucose (Isotope-Cambridge) for ¹⁵N/¹³C labelling respectively. The Nogo peptides Nogo-A (97-107), Nogo-A (141-150) and Nogo-A (171-181) were chemically synthesised using the standard Fmoc method and were purified by HPLC on a reverse-phase C18 column (Vydac). The identities of all proteins and peptides described above were verified by MALDI-TOF mass spectrometry (ABI Voyager-DE™ Biospectrometry™ workstation).

### Example 2

### ITC, NMR Experiments and Structure Determination

All calorimetric titrations were performed on a Microcal VP ITC machine (Microcal, USA). The protein/peptide samples were dissolved in the 50 mM Tris buffer (pH 7.0), with 3 mM 2-mercaptoethanol. After centrifugation for 15 min, the samples were degassed for 15 min to avoid the bubble formation. The Grb4 SH3 samples with a concentration of 0.025 or 0.05 mM were placed in the 1.4 ml reaction cell and 1.0 mM Nogo samples were loaded into the 296.4 ml injection syringe. All titrations were performed at 25° C. and the obtained data was fitted using the ORIGIN software (Originlab Corporation). NMR experiments were performed on a 500 MHz Bruker DRX spectrometer equipped with an actively shielded cryoprobe and an 800 MHz Bruker Avance spectrometer (Sattler, M., et al., (1999) Prog. NMR Spectrosc. 34, 93-158.). NMR data was processed with NMRPipe (Delaglio, F et al., (1995) J. Biomol. NMR 6, 277-293) and analysed with NMRView (Johnson, B. A. & Blevins, R. A. (1994). J. Biomol. NMR 4, 603-614.).

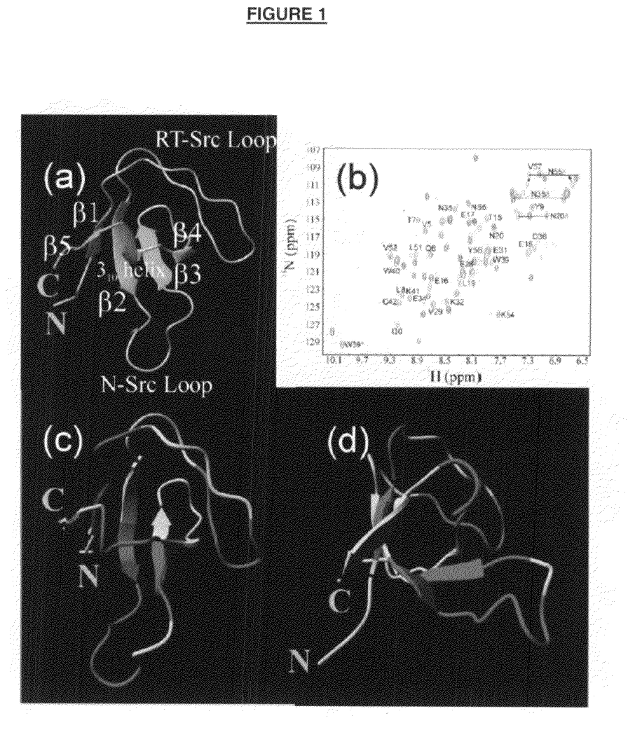

The ¹H-¹⁵N HSQC titration experiments were conducted according to our previous procedure (Song J. (2003) J Biol Chem. 278, 24714-20). For structure determination, backbone sequential assignments were achieved by analysing HNCACB, CBCA(CO)NH, HNCO experiments while side chain assignments were obtained from (H)CC(CO)NH, H(CCO)NH, HCCH-TOCSY, ¹⁵N-edited TOCSY-HSQC and NOESY-HSQC. Structure determination of the third Grb4 SH3 domain was conducted by a standard CNS (Crystallography and NMR System) protocol as described in our previous publication (Li M, et al., 2004, Eur J. Biochem. 271, 3512-22) with NOE constraints derived from ¹⁵N- and ¹³C-NOESY spectra and dihedral angle constraints from TALOS prediction using 5 different chemical shifts. The accepted NMR structure with the lowest energy was selected for analysis and manipulation to generate FIG. 1 by use of MolMol graphic software (Koradi, R., et al., (1996) J. Mol. Graphics. 14, 51-55.).

### Example 3

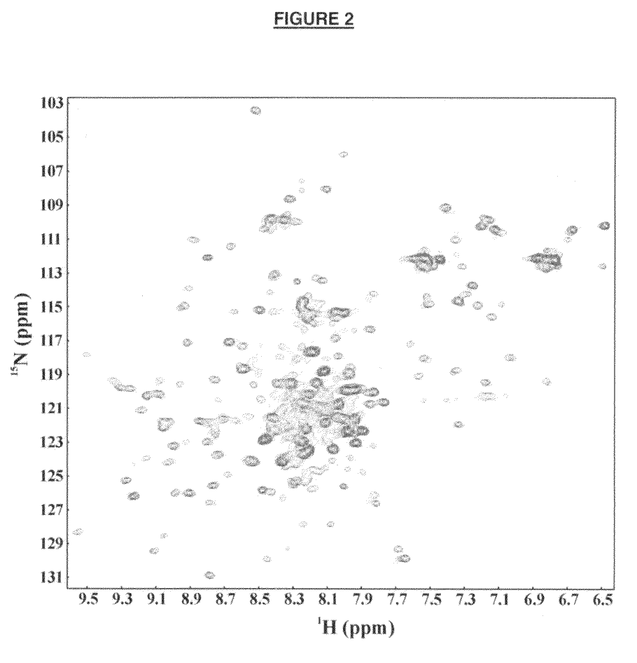

The present inventors also determined whether a cross-talk existed between Nogo and Eph receptor-ephrinB mediated signalling networks, and whether Grb4 was the convergence point. A close examination of Nogo-A sequence revealed that the N-terminal 200 residues of the human Nogo-A, also an equivalent of the N-terminal part of the human Nogo-B, was extremely rich of proline residues which contained 47 proline in total and thus served as ligands for binding to the Grb4 SH3 domains. To test this, as described in the previous examples, express Nogo-A (1-200) and Grb4 (115-257) containing the second and third SH3 domains were expressed and subsequently ¹H-¹⁵N NMR HSQC titration was used to study their binding interaction because NMR HSQC titration represents a very sensitive and quick way to map out binding interactions even with a weak affinity. The titration results showed that significant shifts of the HSQC peaks of the 15N-labeled Grb4 (115-257) were induced by addition of the four-fold excess of the unlabelled Nogo-A (1-200)(see FIG. 2), clearly indicating a binding interaction between Nogo-A (1-200) and Grb4 (115-257).

### Example 4

Both Nogo-A (1-200) and Grb4 (115-257) were further dissected into different Nogo fragments and two different SH2 domains based on sequence alignment as detailed in Table 1 and the interactions among them were studied first by NMR HSQC titration followed by isothermal titration calorimetry (ITC) to obtain thermodynamic parameters of the binding interaction. As presented in Table 2, two complete sets of thermodynamic parameters were derived from ITC measurements, one set for the interaction between Nogo-A (32-200) and the third Grb4 SH3 domain and another set for the interaction between the 11-residue Nogo-A (171-181) and the third Grb4 SH3 domain. The binding affinity between the third Grb4 SH3 domain and the 11-residue peptide Nogo-A (171-181) was about 30-fold higher than of peptide Nogo-A (32-200), indicating that the derived 11-residue peptide is a potent ligand which might efficiently intervene in the Nogo-Grb4 interaction. Many factors account for this observation. One possibility is that in the Nogo-A (32-200) protein, this 11-residue binding region is less accessible. Alternatively, the presence of other regions without any significant contribution to the binding will unavoidably reduce the binding affinity. Strikingly, if compared with other reported SH3-ligand interactions, the binding interaction with the Kd of 2.8 μM between the 11-residue Nogo-A (171-181) and the third SH3 domain should be ranked into the high-affinity category and also be considered to have a very high specificity because this peptide showed no significant binding with the second Grb4 SH3 domain.

In this regard, this 11-residue peptide may also be used as a mimetic to directly interfere in the cross-communication between Nogo and ephrin signalling pathways to achieve therapeutic effects. Moreover, it also serves as an invaluable template for further design of medicine beads.

To further validate the identified binding interaction, we determined the solution structure of the third Grb4 SH3 domain by use of heteronuclear tripleresonance NMR methodology as described briefly in Examples 1 and 2. The NMR structure of the third Grb4 SH3 domain, as seen in FIG. 1a, has a typical SH3 fold composed of five β-strands arranged into two sheets packed at right angle (Musacchio A. (2002) Adv Protein Chem. 61, 211-68; Larson S M, Davidson A R (2000) Protein Sci. 9, 2170-80). Nevertheless, it appears that the third Grb4 SH3 domain has a longer N-Src loop as compared with other SH3 domain structures; which may contribute to its unique binding specificity. Most importantly, the availability of the three dimensional structure allowed us to map out the binding interface between Nogo-A (171-181) and the third Grb4 SH3 domain by use of NMR HSQC titration. As seen in FIG. 1b, upon addition of a two-fold excess of the Nogo-A (171-181) peptide, ˜50% HSQC peaks arising from the ¹⁵N-labeled SH3 domain underwent a significant shift. Some cross peaks, such as the cross peak corresponding to the side chain of Trp39, disappeared completely probably due to the binding-induced conformational exchange on μs and ms time scales. The residues with significant HSQC peak shifts were thus mapped back to the three-dimensional structure of the SH3 domain (FIG. 1c and 1d). The SH3 domain regions with significant perturbations included the RT-Src loop, N-Src loop, the absolutely-conserved 39WW40 motif and the 310-helix over residues 53PKNY56 which have all been previously established to be the critical components constituting the SH3 ligand binding surface. In conclusion, these results therefore defined an atomic-resolution binding interface for the specific interaction between the 11-residue Nogo-A (171-181) peptide and the third Grb4 SH3 domain.

TABLE 1
Binding interactions between the human Nogo and Grb4 fragments
monitored by NMR and isothermal titration calorimetry.

| | | Binding | Binding |
|---|---|---|---|
| | | detected | detected |
| Nogo-A Fragment | Grb4 Fragment^{a} | by NMR | by ITC |
| Nogo-A | SH3_23 | − | \^{b} |
| (1-48) | SH3_2 | − | \ |
| | SH3_3 | − | \ |
| Nogo-A | SH3_23 | − | \ |
| (32-85) | SH3_2 | − | \ |
| | SH3_3 | − | \ |
| Nogo-A | SH3_23 | + | \ |
| (1-200) | SH3_2 | Very weak | \ |
| | SH3_3 | + | \ |
| Nogo-A | SH3_23 | + | Very weak |
| (32-200) | SH3_2 | Very weak | Very weak |
| | SH3_3 | + | + |
| Nogo-A | SH3_23 | \ | \ |
| (97-107) | SH3_2 | − | \ |
| Ac-APPVAPERQPS-NH₂ | SH3_3 | − | \ |
| Nogo-A | SH3_23 | \ | \ |
| (141-150) | SH3_2 | − | − |
| Ac-ARPPPPPPAS-NH₂ | SH3_3 | + | Very weak |
| Nogo-A | SH3_23 | \ | \ |
| (171-181) | SH3_2 | − | − |
| Ac-STPAAPKRRGS-NH₂ | SH3_3 | + | + |

^{a}Grb4 fragments used in the experiments included: SH3_23 containing the second and third SH3 domains corresponding to Grb4 residues 115-257; SH3_2 containing the second SH3 domain corresponding to Grb4 residues 115-170; and SH3_3 containing the third SH3 domain corresponding to Grb4 residues 199-257.
^{b}not tested.

TABLE 2
Thermodynamic parameters of the binding interaction between Nogo peptides and
Grb4 SH3 domains measured by isothermal titration calorimetry at 25° C.

| | | | Kₐ | K_{d} | Stoichiometry | ΔS | ΔH |
|---|---|---|---|---|---|---|---|
| Syringe | Cell | Buffer | (M⁻¹) | (μM) | n | (cal/mol) | (cal/mol) |
| Nogo-A | SH3_3 | Tris, | 1.15E4 ± 2.2E3 | 87 | 1.13 ± 0.096 | 252 | 6.960E4 ± 1.06E3 |
| [32-200] | (25 μM) | pH 7.0 | | | | | |
| Nogo-A | SH3_3 | Tris, | 3.57E5 ± 2.1E4 | 2.8 | 0.95 ± 0.068 | 14.3 | −3,310 ± 499 |
| [171-181] | (50 μM) | pH 7.0 | | | | | |

### REFERENCES

Acevedo L, et al., (2004) Nat. Med. 10, 382-8
Cowan C A, Henkemeyer M. (2002) Trends Cell Biol. 12, 339-46
Cowan C A, Henkemeyer M. (2001) Nature. 413, 174-9
Delaglio, F et al., (1995) J. Biomol. NMR 6, 277-293
Fournier, A. E. et al. (2001) Nature 409:341-346
Fournier A E, Strittmatter S M. (2001) Curr Opin Neurobiol. 11, 89-94
Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY
He W, et al., (2004) Nat. Med. 10, 959-65
Karnezis T, et al., (2004) Nat. Neurosci. 7, 736-44
Koradi, R., et al., (1996) J. Mol. Graphics. 14, 51-55
Johnson, B. A. & Blevins, R. A. (1994). J. Biomol. NMR 4, 603-614
Larson S M, Davidson A R (2000) Protein Sci. 9, 2170-80
Lee D H, et al., (2003) Nat Rev Drug Discov. 2, 872-8
Li M, et al., (2004) Eur J. Biochem. 271, 3512-22
Li Q, et al., (2001) Oncogene. 20, 3929-36)
Li W, et al., (2001) Oncogene. 20, 6403-17
Musacchio A. (2002) Adv Protein Chem. 61, 211-68
Oertle T, Schwab M E. (2003) Trends Cell Biol. 13, 187-94
Sambrook and Russel, Molecular Cloning, A laboratory manual, Cold Spring Harbour Laboratory Press, 2001
Sandvig A, Berry M, Barrett L B, Butt A, Logan A. (2004) Glia. 46, 225-51
Sattler, M., et al., (1999) Prog. NMR Spectrosc. 34, 93-158
Song J. (2003) J Biol. Chem. 278, 24714-20
Tu Y, Li F, Wu C. (1998) Mol Biol Cell. 9, 3367-82

1-33. (canceled)

34. An isolated polypeptide, wherein the polypeptide is selected from the group consisting of:
(a) a fragment of Nogo-A protein, the fragment being capable of inhibiting the interaction between Nogo-ephrinB and/or Nogo-Grb4 third SH3 domain;
(b) a polypeptide comprising amino acids 1-200 of human Nogo-A or a biologically active fragment thereof capable of inhibiting the interaction between Nogo-ephrinB and/or Nogo-Grb4 third SH3 domain;
(c) a fragment of Nogo-A protein, the fragment comprising the amino acids 1-200 of SEQ ID NO:30;
(d) a polypeptide comprising amino acids 32-200 of human Nogo-A or a biologically active fragment thereof capable of inhibiting the interaction between Nogo-ephrinB and/or Nogo-Grb4 third SH3 domain;
(e) a fragment of Nogo-A protein, the fragment comprising amino acids 32-200 of SEQ ID NO:30;
(f) a polypeptide fragment comprising the amino acid sequence X³X⁵[P]_{z}X³X¹, the fragment being capable of inhibiting the interaction between Nogo-ephrinB and/or Nogo-Grb4 third SH3 domain;
(g) a polypeptide fragment comprising the amino acid sequence of SEQ ID NO:3, the fragment capable of inhibiting the interaction between Nogo-ephrinB and/or Nogo-Grb4 third SH3 domain;
(h) a polypeptide having the amino acid sequence of [Σ]ₙX³X⁵[P]_{z}X³X¹[U]ₘ;
(i) a polypeptide fragment comprising the amino acid sequence X¹X²PX³X³PX⁴X⁵X⁵X⁶X¹, the fragment capable of inhibiting the interaction between Nogo-ephrinB and/or Nogo-Grb4 third SH3 domain;
a polypeptide fragment comprising the amino acid sequence SEQ ID NO: 1, the fragment capable of inhibiting the interaction between Nogo-ephrinB and/or Nogo-Grb4 third SH3 domain; and
(k) a polypeptide having the amino acid sequence of [Σ]ₙX¹X²PX³X³PX⁴X⁵X⁵X⁶X¹[U]ₘ or a biologically active fragment thereof,
wherein each of X¹ independently is any hydrophilic amino acid; X² is any hydrophilic amino acid; each of X³ independently is a hydrophobic amino acid or Y; X⁴ is any basic amino acid; each of X⁵ independently is any basic amino acid; X⁶ is any hydrophilic amino acid; [Σ] is any amino acid, and n is an integer from 0 to 20; [U] is any amino acid, and m is an integer from 0 to 20; and z is an integer from 1 to 6.

35. The polypeptide according to claim 34, wherein the polypeptide is X¹X²PX³X³PX⁴X⁵X⁵X⁶X¹.

36. The polypeptide according to claim 34, wherein the polypeptide has the amino acid sequence of SEQ ID NO: 1 or a biologically active fragment thereof.

37. The polypeptide according to claim 34, wherein the polypeptide is X³X⁵ PPPPPPX³X¹.

38. The polypeptide according to claim 34, wherein the polypeptide has the amino acid sequence of SEQ ID NO:3 or a biologically active fragment thereof.

39. An isolated polynucleotide encoding a polypeptide according to claim 34.

40. The polynucleotide according to claim 39, wherein the polynucleotide encodes a polypeptide having the amino acid sequence of SEQ ID NO: 1 or SEQ ID NO:3 a biologically active fragment thereof.

41. A vector comprising the polynucleotide according to claim 39.

42. A cell transformed with the polynucleotide according to claim 39.

43. A cell transformed with the vector according to claim 41.

44. A method of producing a polypeptide according to claim 34, wherein the method comprises the steps of:
(a) culturing a cell under conditions suitable for expression of the polypeptide or fragment thereof, wherein said cell is transformed with a vector comprising a polynucleotide encoding the polypeptide or fragment thereof; and
(b) recovering the polypeptide or fragment thereof so expressed.

45. The method according to claim 44, wherein the polypeptide comprises the amino acid sequence of SEQ ID NO: 1 or a biologically active fragment thereof.

46. The method according to claim 44, wherein the polypeptide comprises the amino acid sequence of SEQ ID NO:3 or a biologically active fragment thereof.

47. A composition comprising a polypeptide according to claim 34.

48. A composition according to claim 47, wherein the polypeptide comprises the amino acid sequence of SEQ ID NO: 1; SEQ ID NO:3; or a biologically active fragment thereof.

49. The composition according to claim 47, further comprising a pharmaceutically acceptable diluent, carrier and/or excipient.

50. A method of modulating the Nogo-ephrinB and/or Nogo-Grb4 third SH3 domain interaction comprising adding the polypeptide or fragment thereof according to claim 34 to a cell in vitro or in vivo.

51. The method according to claim 50, wherein the polypeptide or fragment thereof inhibits the interaction between Nogo-ephrinB and/or Nogo-Grb4 third SH3 domain.

52. A method for treating a neuronal- and/or angiogenesis-associated condition comprising administering to a subject in need a composition comprising a therapeutically effective amount of a polypeptide or a biologically active fragment thereof, the polypeptide or the fragment thereof capable of modulating the interaction between Nogo-ephrinB and/or Nogo-Grb4 third SH3 domain.

53. The method according to claim 52, wherein the polypeptide or fragment thereof is capable of inhibiting the interaction between Nogo-ephrinB and/or Nogo-Grb4 third SH3 domain.

54. The method according to claim 52, wherein the polypeptide is a polypeptide having a binding affinity to Grb4.

55. The method according to claim 52, wherein the polypeptide is a polypeptide according to any one of claims 1 to 5.

56. The method according to claim 52, wherein the polypeptide comprises the amino acid sequence of SEQ ID NO:1; SEQ ID NO:3; or a biologically fragment thereof.

57. The method according to claim 52, wherein the neuronal- and/or angiogenesis-associated condition is selected from the group consisting of: control of neuronal development, neuronal regeneration, angiogenesis, inhibition of neurite growth, regulating autoimmune-mediated demyelination, regulator of vascular remodelling, apoptosis-inducer.

58. The method according to claim 52, wherein the subject is a mammal.

59. The method according to claim 52, wherein the subject is a human.

60. A method of screening a compound for effectiveness as an agonist or as an antagonist of a polypeptide or fragment thereof according to claim 34, the method comprising:
(a) exposing a sample comprising a polypeptide or fragment thereof according to claim 34 to a compound; and
(b) detecting agonist or antagonist activity in the sample.

61. The method according to claim 60, wherein the polypeptide comprises the amino acid sequence of SEQ ID NO: 1; SEQ ID NO:3; or a biologically fragment thereof.

62. A method of screening a compound for effectiveness as a modulator of Nogo-ephrinB and/or Nogo-Grb4 third SH3 domain interaction, the method comprising:
(a) adding a test compound to a cell in vitro or in vivo; and
(b) detecting the modulating activity of the test compound.

63. A method of screening a compound for effectiveness as a modulator of Nogo-ephrinB and/or Nogo-Grb4 third SH3 domain interaction, the method comprising:
(a) adding a test compound to a cell in vitro or in vivo;
(b) detecting the modulating activity of the test compound;
(c) adding a polypeptide or fragment thereof comprising the amino acid sequence of according to claim 34;
(d) detecting the modulating activity of the polypeptide or fragment thereof; and
(e) comparing the modulating activity of the test compound with the modulating activity of the polypeptide or a fragment thereof.

64. The method according to claim 62, wherein the polypeptide comprises the amino acid sequence of SEQ ID NO: 1; SEQ ID NO:3, or a biologically active fragment thereof.


SEQUENCE LISTING (30 sequences)

SEQ ID NO: 1 - protein, 11 aa, Human Nogo (fragment 171-181 of Nogo-A)
STPAAPKRRGS

SEQ ID NO: 2 - protein, 11 aa, Human Nogo (fragment  97-107 of Nogo-A)
APPVAPERQPS

SEQ ID NO: 3 - protein, 10 aa, Human Nogo (fragment 141-150 of Nogo-A)
ARPPPPPPAS

SEQ ID NO: 4 - DNA, 54 bp, Artificial
  Primer for human Grb4 residues 5-61
gtgattgtgattgcgaaatgggattataccgcgcagcaggatcaggaactggat

SEQ ID NO: 5 - DNA, 54 bp, Artificial
  Primer for human Grb4 residues 5-61
atcatccagcagccacagacgttcgttcacttttttaatatccagttcctgatc

SEQ ID NO: 6 - DNA, 54 bp, Artificial
  Primer for human Grb4 residues 5-61
tggctgctggatgatagcaaaacctggtggcgtgtgcgtaacgcggcgaaccgt

SEQ ID NO: 7 - DNA, 54 bp, Artificial
  Primer for human Grb4 residue 5-61
gtttttacgttccacatagttgctcggcacatagccggtacggttcgccgcgtt

SEQ ID NO: 8 - DNA, 36 bp, Artificial
  Primer for human Grb4 residues 5-61
cagaacgcggggggatccgtgattgtgattgcgaaa

SEQ ID NO: 9 - DNA, 36 bp, Artificial
  Primer for human Grb4 residues 5-61
cagaacccgctcgagtcagtttttacgttccacata

SEQ ID NO: 10 - DNA, 54 bp, Artificial
  Primer for human Grb4 residues 115-171
attccggcgtttgtgaaatttgcgtatgtggcggaacgtgaagatgaactgagc

SEQ ID NO: 11 - DNA, 54 bp, Artificial
  Primer for human Grb4 residues 115-171
gcatttttccatcacggtcacacggctgcctttcaccaggctcagttcatcttc

SEQ ID NO: 12 - DNA, 54 bp, Artificial
  Primer for human Grb4 residues 115-171
gtgatggaaaaatgcagcgatggctggtggcgtggcagctataacggccagatt

SEQ ID NO: 13 - DNA, 54 bp, Artificial
  Primer for human Grb4 residues 115-171
atccacttcttccagcacatagttgctcggaaaccagccaatctggccgttata

SEQ ID NO: 14 - DNA, 36 bp, Artificial
  Primer for human Grb4 residues 115-171
cagaacgcggggggatccattccggcgtttgtgaaa

SEQ ID NO: 15 - DNA, 36 bp, Artificial
  Primer for human Grb4 residues 115-171
cagaacccgctcgagtcaatccacttcttccagcac

SEQ ID NO: 16 - DNA, 54 bp, Artificial
  Prmer for human Grb4 residues 199-257
catgtggtgcagaccctgtatccgtttagcagcgtgaccgaagaagaactgaac

SEQ ID NO: 17 - DNA, 54 bp, Artificial
  Primer for human Grb4 residues 199-257
cggtttttcaatcacttccatggtttcgcctttttcaaagttcagttcttcttc

SEQ ID NO: 18 - DNA, 54 bp, Artificial
  Primer for human Grb4 residues 199-257
gtgattgaaaaaccggaaaacgatccggaatggtggaaatgcaaaaacgcgcgt

SEQ ID NO: 19 - DNA, 60 bp, Artificial
  Primer for human Grb4 residues 199-257
gctcagcaccaccacatagtttttcggcaccaggcccacctggccacgcgcgtttttgca

SEQ ID NO: 20 - DNA, 36 bp, Artificial
  Primer for human Grb4 residues 199-257
cagaacgcggggggatcccatgtggtgcagaccctg

SEQ ID NO: 21 - DNA, 36 bp, Artificial
  Primer for human Grb4 residues 199-257
cagaacccgctcgagtcagctcagcaccaccacata

SEQ ID NO: 22 - DNA, 63 bp, Artificial
  Primer
cgcgcctttacgcaggctcagaaagctcgggctttccgccgccgcttcatccacttcttccag

SEQ ID NO: 23 - DNA, 63 bp, Artificial
  Primer
ctgcgtaaaggcgcgagcctgagcaacggccagggcagccgtgtgctgcatgtggtgcagacc

SEQ ID NO: 24 - DNA, 33 bp, Artificial
  Primer for Nogo-A (1-200)
cgcgcgcgcggatccatggaagacctggaccag

SEQ ID NO: 25 - DNA, 36 bp, Artificial
  Primer for Nogo-A (1-200)
cgcgcgcgcctcgagctatatcacaggctcagatgc

SEQ ID NO: 26 - DNA, 30 bp, Artificial
  Primer for Nogo-A (32-85)
cgcgcgcgcggatccgaggacgaggaggaa

SEQ ID NO: 27 - DNA, 36 bp, Artificial
  Primer for Nogo-A (32-85)
cgcgcgcgcctcgagctagaagtcatttccgaagtc

SEQ ID NO: 28 - DNA, 33 bp, Artificial
  Primer for Nogo-A (32-200)
cgcgcgcgcggatccttcgtgccgccggcgccc

SEQ ID NO: 29 - DNA, 36 bp, Artificial; identical to SEQ ID NO: 25
  Primer for Nogo-A (32-200)

SEQ ID NO: 30 - protein, 1192 aa, Homo sapiens (Nogo-A protein) [sequence omitted]